(19) United States
(12) Patent Application Publication
(10) Pub. No.: US 2018/0002171 A1
(43) Pub. Date: Jan. 4, 2018

(54) NANO-BIOREACTOR APPARATUS AND METHOD OF MANIPULATING EXTRACELLULAR METABOLIC SYSTEMS

(71) Applicants: Ghassem Amoabediny, Tehran (IR); Mahdi Pesaran, Tehran (IR)

(72) Inventors: Ghassem Amoabediny, Tehran (IR); Mahdi Pesaran, Tehran (IR)

(21) Appl. No.: 15/604,651

(22) Filed: May 25, 2017

(30) Foreign Application Priority Data
Jun. 29, 2016 (IR) 13955014000300413

(51) Int. Cl. B82Y 5/00; B01L 99/00; C12N 11/02; B01J 19/00; C12M 1/40; C12M 1/00; C12Q 1/02; G01N 35/00

(52) U.S. Cl. CPC B82Y 5/00; C12M 29/00; B01L 99/00; C12Q 1/02; C12N 11/02; B01J 19/0093; C12M 1/40; B01L 2300/0896; G01N 2035/0094

(57) ABSTRACT

A nano-bioreactor, and an enzymatic nano-bioreactor apparatus are disclosed. The enzymatic nano-bioreactor, comprises at least one enzyme, and a cellulose nano-fiber. The enzyme is any one of a urease, a glutamate dehydrogenase, and a glutamine synthetase configured to immobilize on the cellulose nano-fiber. The enzymatic nano-bioreactor apparatus comprises a plurality of input pumps, which is configured to introduce at least one input to a plurality of micro-bioreactors, where connected each other by a connector. The apparatus further comprises, at least one nano-bioreactor placed inside the micro-bioreactor, configured to receive the input, and an outlet configured to extrude a resultant output from the nano-bioreactor to a collector unit. The present also discloses a method for manipulating extracellular metabolic system using the said enzymatic nano-bioreactor apparatus.

### BACKGROUND OF THE INVENTION

Enzyme immobilization is a special technique to solve enzymatic problems, such as stability, reusability, and decline of activity due to inhibition by either medium or products. The immobilization of enzyme is influenced by the properties of support, such as material type, composition, structure, and mechanical properties. Enhanced support properties offer a good mechanical strength, which can contribute to stability and reusability. Nano-sized supports are utilized not only to improve the stability and reusability of the immobilized enzyme but also to overcome their lower immobilized enzyme activity owing to presence of high surface area per volume ratio. In other words, high surface area of nano-sized support provides a high number of functional groups on the surface support.

Generally, the enzymes are immobilized on various nanostructured materials using conventional approaches, like simple adsorption and covalent attachment in nano-biocatalysis process. Nanostructured materials have been used as hosts for enzyme immobilization to improve the performance of enzymes in various biocatalytic processes. Among all nanostructured supports, nanofibers are distinguished as a good support for the immobilization of enzymes due to their high surface area-to-volume ratio and less mass transfer resistance. The recycling of the nanofibers is found to be easier in comparison to the other nanostructure materials. Moreover, the cellulosic nanocomposites have been considered as an innovative solution to produce materials with advanced properties, due to its continuous nature and nano-scaled diameters and high crystallinity.

Enzymes such as urease catalyze the hydrolysis of urea. It is related to protein intake, nitrogen metabolism and excretion. Many applications exist, such as blood detoxification in artificial kidneys, the removal of urea from beverages and foods in food industry, and the reduction of urea content in effluent treatment in agriculture. There are many methods for enzyme immobilization including the adsorption, entrapment, and covalent binding. Among them, the entrapment in nanofiber matrixes has been done by cross-linking reaction between enzyme molecules.

Thus, there is a clear and present need for a novel design and fabrication of an in-vitro enzymatic bioreactor for manipulating extracellular metabolic pathway with the capability of reusability for the urea uptake. There is also a need for a method of using the enzymatic bioreactor for simulating extracellular metabolism uptake, and conversion of urea into glutamic acid.

### SUMMARY OF THE INVENTION

The present invention relates to a nano-bioreactor, an enzymatic nano-bioreactor apparatus and a method for manipulating extracellular metabolic system using the aforementioned enzymatic nano-bioreactor apparatus, according to an embodiment. In an embodiment, an enzymatic nano-bioreactor, comprises at least one enzyme, and a nano-fiber. In one embodiment, the enzyme is a nanoparticle configured to immobilize on the nano-fiber. The enzyme could be any one of a urease, a glutamate dehydrogenase, and a glutamine synthetase. The nano-fiber is a cellulose nano-fiber, or a bacterial cellulose nano-fiber. In one embodiment, the enzyme is immobilized by crosslinking mechanism to the nanofibers. In another embodiment, the enzyme is immobilized by covalent reaction to the nanofibers. In another embodiment, the enzyme is immobilized by both crosslinking to the nanofibers and by covalent reaction to the nanofibers.

In an embodiment, the enzyme comprises urease nanoparticle configured to immobilize on the nano-fiber to form a urease nano-bioreactor. In one embodiment, the enzyme comprises glutamate dehydrogenase nanoparticle configured to immobilize on the nano-fiber to form a glutamate dehydrogenase nano-bioreactor. In another embodiment, the enzyme comprises glutamine synthetase nanoparticle configured to immobilize on the nano-fiber to form a glutamine synthetase nano-bioreactor. In an embodiment, the optimized pH and temperature for immobilizing urease on the nano-fiber is 6.5 and 50° C. In one embodiment, the optimized pH and temperature for immobilizing glutamate dehydrogenase on the nano-fiber is 8.5 and 50° C. In another embodiment, the optimized pH and temperature for immobilizing glutamine synthetase on the nano-fiber is 7.5 and 60° C.

In an embodiment, an enzymatic nano-bioreactor apparatus comprises a plurality of input pumps, which is configured to introduce at least one input to a plurality of micro-bioreactors. In one embodiment, the said micro-bioreactors connected each other by a connector, for example silicon connector. The apparatus further comprises, at least one nano-bioreactor placed inside the micro-bioreactor, configured to receive the input. In an embodiment, the nano-bioreactor comprises at least one enzyme, and a nano-fiber, where the enzyme is a nanoparticle configured to immobilize on the nano-fiber, and an outlet configured to extrude a resultant output from the nano-bioreactor to a collector unit. In one embodiment, the input is urea and the resultant output is glutamine.

In an embodiment, the input urea is converted to ammonium by the urease nano-bioreactor. In one embodiment, the ammonium is converted to L-Glutamate by the glutamate dehydrogenase nano-bioreactor. In another embodiment, the L-Glutamate is converted to output glutamine by the glutamine synthetase nano-bioreactors.

In an embodiment, a method for manipulating extracellular metabolic system, comprises introducing an input via a plurality of input pumps to one or more micro-bioreactors connected each other by a connector. In one step, input is received by a first nano-bioreactor placed inside the first micro-bioreactor to produce first output. In another step, the first output is received by a second nano-bioreactor placed inside the second micro-bioreactor to produce second output. In next step, the second input is received by a third nano-bioreactor placed inside the third micro-bioreactor to produce final output. In final step, the final output is extruded and collected via an outlet to a collector unit. In one embodiment, the first output is ammonium, the second output is L-Glutamate, and the final output is glutamine.

In one embodiment, the optimized pH and temperature for immobilizing urease on the nano-fiber is 6.5 and about 50° C., and the optimized pH and temperature for immobilizing glutamate dehydrogenase on the nano-fiber is 8.5 and about 50° C., and the optimized pH and temperature for immobilizing glutamine synthetase on the nano-fiber is 7.5 and about 60° C. In another embodiment, the input urea is converted to ammonium by the urease nano-bioreactor, and the ammonium is converted to L-Glutamate by the glutamate dehydrogenase nano-bioreactor, and the L-Glutamate is converted to output glutamine by the glutamine synthetase nano-bioreactor.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

FIG. 1 illustrates an enzymatic nano-bioreactor apparatus to manipulate extracellular metabolic pathway for urea uptake according to an embodiment;

FIG. 2 shows a micro-bioreactor implemented with the enzymatic nano-bioreactor according to an embodiment;

FIG. 3 shows a method for manipulating extracellular metabolic system using the enzymatic nano-bioreactor apparatus according to an embodiment;

FIG. 4 is a graph illustrating an effect of pH on the free and immobilized Urease activity;

FIG. 5 is a graph illustrating an effect of temperature on the free and immobilized Urease activity;

FIG. 6 is a graph illustrating operational stability of Immobilized Urease;

FIG. 7 is a graph illustrating operational reusability of Immobilized Urease;

FIG. 8 is a flowchart illustrating a method of GDH immobilization on BCN;

FIG. 9 is a graph illustrating an effect of pH on the free and immobilized glutamate dehydrogenase activity;

FIG. 10 is a graph illustrating an effect of temperature on the free and immobilized glutamate dehydrogenase activity;

FIG. 11 is a graph illustrating operational stability of immobilized glutamate dehydrogenase on BCN;

FIG. 12 is a graph illustrating operational reusability of immobilized glutamate dehydrogenase on BCN;

FIG. 13 is a graph illustrating an effect of pH on Immobilized Glutamine Synthetase on a Cellulose Nanofiber Matrix;

FIG. 14 is a graph illustrating an effect of temperature on Immobilized Glutamine Synthetase on a Cellulose Nanofiber Matrix;

FIG. 15 is a graph illustrating operational stability of Immobilized Glutamine Synthetase on a Cellulose Nanofiber Matrix;

FIG. 16 is a graph illustrating operational reusability of Immobilized Glutamine Synthetase on a Cellulose Nanofiber Matrix;

FIG. 17 illustrates a schematic diagram of designed vessel; and

FIG. 18 illustrates a schematic diagram of manipulated extracellular metabolic system.

### DETAILED DESCRIPTION

The present invention generally relates to a bioreactor, and more particularly relates to a nano-bioreactor and apparatus, used for the simulation of cellular metabolism uptake and conversion of urea into glutamic acid. The embodiments herein more particularly relates to a method of using the nano-bioreactor apparatus implemented with a plurality of immobilized enzymes for the simulation of the extra-cellular metabolism uptake and conversion of urea into glutamic acid.

Metabolic paths are the most general way for elimination and production of natural materials by nature. Enzymes play key role amongst these methods. However, instability of the enzymes is a critical problem. Immobilization on adequate matrix is an effective solution to overcome this problem. In this manner, due to recent developments in nano industries and nano structures with unique properties, utilization of these materials as a immobilization matrix according to their high surface to volume ratio and high mass transfer ability, results in higher stability and improvement in immobilized enzyme activity.

A description of embodiments of the present invention will now be given with reference to the figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

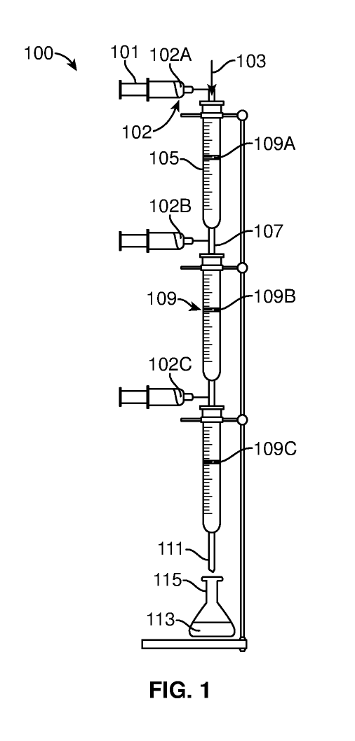

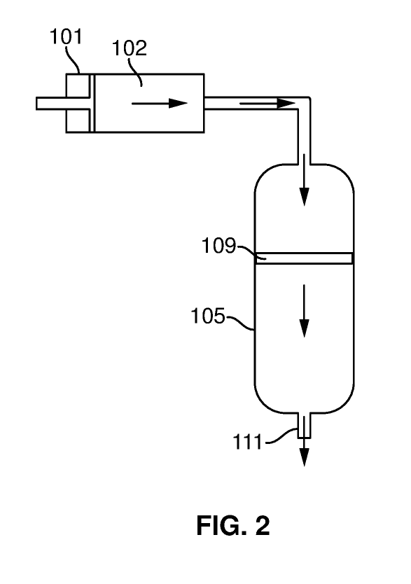

According to an embodiment of the invention, a nano-bioreactor, an enzymatic nano-bioreactor apparatus and a method for manipulating extracellular metabolic system using the said enzymatic nano-bioreactor apparatus are disclosed. Referring to FIGS. 1 and 2, the enzymatic nano-bioreactor apparatus 100 comprises a plurality of input pumps 101, which is configured to introduce at least one input 103 to a plurality of micro-bioreactors 105. In one embodiment, the input pump 101 could be a syringe pump, where one or more reactants or substrates 102 could be introduced into the micro-bioreactors 105. In some embodiments, the substrates 102 are urease substrate 102A, glutamate dehydrogenase substrate 102B, and glutamine synthetase substrate 102C.

In one embodiment, the said micro-bioreactors 105 connected each other by a connector 107, for example silicon connector. The apparatus 100 further comprises, at least one nano-bioreactor 109 placed inside the micro-bioreactor 105, configured to receive the input 103. In an embodiment, the nano-bioreactor 109 comprises at least one enzyme, and a nano-fiber, where the enzyme is a nanoparticle configured to immobilize on the nano-fiber, and an outlet 111 configured to extrude a resultant output 113 from the nano-bioreactor 109 to a collector unit 115. In one embodiment, the input 103 is urea and the resultant output 113 is glutamine.

In an embodiment, the input urea 103 is converted to ammonium by the urease nano-bioreactor 109A. In one embodiment, the ammonium is converted to L-Glutamate by the glutamate dehydrogenase nano-bioreactor 109B. In another embodiment, the L-Glutamate is converted to the resultant output glutamine by the glutamine synthetase nano-bioreactors 109C.

Referring to FIG. 2, an enzymatic nano-bioreactor 109, comprises at least one enzyme, and a nano-fiber. In one embodiment, the enzyme is a nanoparticle configured to immobilize on the nano-fiber. The enzyme could be any one of a urease, a glutamate dehydrogenase, and a glutamine synthetase. The nano-fiber is a cellulose nano-fiber, or a bacterial cellulose nano-fiber. In one embodiment, the enzyme is immobilized by covalent and then crosslinking mechanism to the nanofibers.

In an embodiment, the enzyme comprises urease nanoparticle configured to immobilize on the nano-fiber to form a urease nano-bioreactor 109A. In one embodiment, the enzyme comprises glutamate dehydrogenase nanoparticle configured to immobilize on the nano-fiber to form a glutamate dehydrogenase nano-bioreactor 109B. In another embodiment, the enzyme comprises glutamine synthetase nanoparticle configured to immobilize on the nano-fiber to form a glutamine synthetase nano-bioreactor 109C. In an embodiment, the optimized pH and temperature for immobilizing urease on the nano-fiber is 6.5 and 50° C. In one embodiment, the optimized pH and temperature for immobilizing glutamate dehydrogenase on the nano-fiber is 8.5 and 50° C. In another embodiment, the optimized pH and temperature for immobilizing glutamine synthetase on the nano-fiber is 7.5 and 60° C.

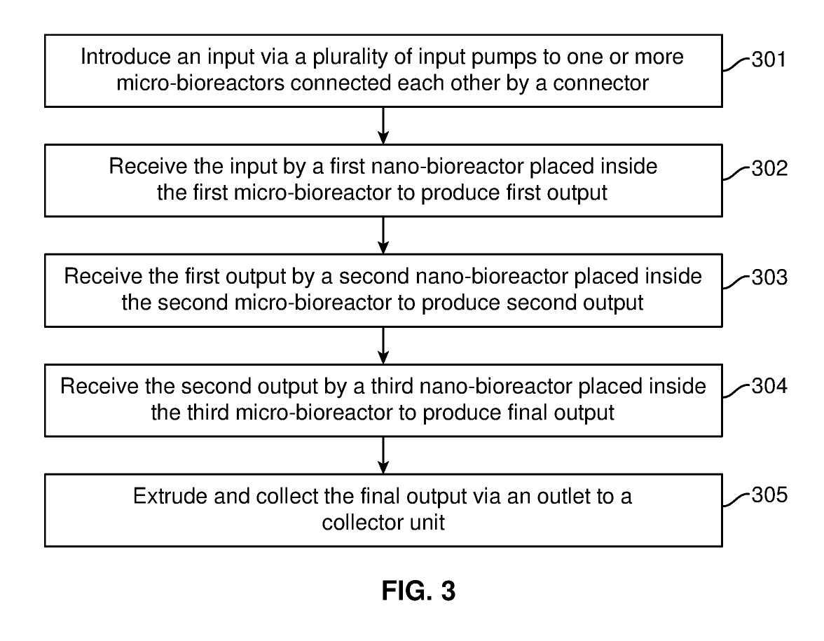

Referring to FIG. 3, a method for manipulating extracellular metabolic system, is illustrated from step 301 to 305. In step 301, the method according to the invention, comprises introducing an input via a plurality of input pumps to one or more micro-bioreactors connected each other by a connector. In step 302, input is received by a first nano-bioreactor placed inside the first micro-bioreactor to produce first output. In step 303, the first output is received by a second nano-bioreactor placed inside the second micro-bioreactor to produce second output. In step 304, the second input is received by a third nano-bioreactor placed inside the third micro-bioreactor to produce final output. In final step 305, the final output is extruded and collected via an outlet to a collector unit. In one embodiment, the first output is ammonium, the second output is L-Glutamate, and the final output is glutamine.

One aspect of the present disclosure is directed to an enzymatic nano-bioreactor, comprising at least one enzyme, and a nano-fiber, wherein the enzyme is a nanoparticle configured to immobilize on the nano-fiber. In one embodiment, the enzyme is any one of a urease, a glutamate dehydrogenase, and a glutamine synthetase. In one embodiment, the nano-fiber is a cellulose nano-fiber. In another embodiment, the nano-fiber is a bacterial cellulose nano-fiber. In one embodiment, the enzyme is immobilized by crosslinking mechanism to the nanofibers. In another embodiment, the enzyme is immobilized by covalent reaction to the nanofibers. In one embodiment, the enzyme comprising urease nanoparticle configured to immobilize on the nano-fiber to form a urease nano-bioreactor. In another embodiment, the enzyme comprising glutamate dehydrogenase nanoparticle is configured to immobilize on the nano-fiber to form a glutamate dehydrogenase nano-bioreactor.

In one embodiment of the enzymatic nano-bioreactors, the enzyme comprising glutamine synthetase nanoparticle is configured to immobilize on the nano-fiber to form a glutamine synthetase nano-bioreactor. In one embodiment, the optimized pH and temperature for immobilizing urease on the nano-fiber is 6.5 and about 50° C. In one embodiment, the optimized pH and temperature for immobilizing glutamate dehydrogenase on the nano-fiber is 8.5 and about 50° C. In one embodiment, the optimized pH and temperature for immobilizing glutamine synthetase on the nano-fiber is 7.5 and about 60° C.

Henceforth, immobilization of each enzyme and proper selection of nanofiber material, are used effectively for simulating extracellular metabolic pathway of urea uptake. Triple immobilized enzyme on cellulose nanofiber system is placed into the micro-bioreactor or micro-container. The micro-bioreactor may include one or more inlet or entrance, and discharge for supplying substrate or react.

All these substrate are injected through syringe pump with a predetermined velocity. The input and output velocity of the substrate or reactor materials are directly related to enzyme reaction velocity. The enzymes are immobilized aforementioned nanofiber, a nano-bioreactor has been constructed and placed into a micro-bioreactor. Usage of nanofibers has solved dissipation problem of nanoparticles in media and facilitate the way to gather them from media for further reactions. Moreover, the operation cost of nanofiber production is lower compared to the nanoparticles. Synthetased nanofibers are porous and have shorter penetration path and shorter access path to outer space.

Another aspect of the present disclosure is directed to an enzymatic nano-bioreactor apparatus, comprising: (a) a plurality of input pumps configured to introduce at least one input to a plurality of micro-bioreactors connected each other by a connector, and (b) at least one nano-bioreactor placed inside the micro-bioreactor, configured to receive the input, wherein the nano-bioreactor comprising: (i) at least one enzyme, and (ii) a nano-fiber, wherein the enzyme is a nanoparticle configured to immobilize on the nano-fiber, and an outlet configured to extrude a resultant output from the nano-bioreactor to a collector unit.

In one embodiment of the enzymatic nano-bioreactor apparatus, the input is urea and the resultant output is glutamine. In another embodiment, the plurality of micro-bioreactors are connected to each other by a silicon connector. In another embodiment, the enzyme is any one of a urease, a glutamate dehydrogenase, and a glutamine synthetase. In one embodiment, the nano-fiber is a cellulose nano-fiber. In another embodiment, the nano-fiber is a bacterial cellulose nano-fiber. In one embodiment, the enzyme comprising urease nanoparticle configured to immobilize on the nano-fiber to form a urease nano-bioreactor. In one embodiment, the enzyme comprising glutamate dehydrogenase nanoparticle configured to immobilize on the nano-fiber to form a glutamate dehydrogenase nano-bioreactor.

In one example, the enzyme comprising glutamine synthetase nanoparticle is configured to immobilize on the nano-fiber to form a glutamine synthetase nano-bioreactor. In one embodiment, the enzyme is immobilized by crosslinking mechanism to the nanofibers. In one embodiment, the enzyme is immobilized by covalent reaction to the nanofibers. In one embodiment, the input urea is converted to ammonium by the urease nano-bioreactor. In a related embodiment, the ammonium is converted to L-Glutamate by the glutamate dehydrogenase nano-bioreactor. In a related embodiment, the L-Glutamate is converted to output glutamine by the glutamine synthetase nano-bioreactor.

Another aspect of the present disclosure is directed to a method for manipulating extracellular metabolic system. This method comprises (a) introducing an input via a plurality of input pumps to one or more micro-bioreactors connected each other by a connector; (b) receiving the input by a first nano-bioreactor placed inside the first micro-bioreactor to produce first output; (c) receiving the first output by a second nano-bioreactor placed inside the second micro-bioreactor to produce second output; (d) receiving the second output by a third nano-bioreactor placed inside the third micro-bioreactor to produce final output, and (e) extruding and collecting the final output via an outlet to a collector unit. In one embodiment, the manipulation is the adjusting of the extracellular metabolic system.

In one embodiment, the input is urea and the resultant output is glutamine. In another embodiment, the plurality of micro-bioreactors are connected each other by a silicon connector. In another embodiment, the first nano-bioreactor comprises urease enzyme nanoparticles immobilized on a nano-fiber to form a urease nano-bioreactor. In one embodiment, the second nano-bioreactor comprises glutamate dehydrogenase enzyme nanoparticles immobilized on the nano-fiber to form a glutamate dehydrogenase nano-bioreactor. In one embodiment, the third nano-bioreactor comprises glutamine synthetase enzyme nanoparticles immobilized on the nano-fiber to form a glutamine synthetase nano-bioreactor. In one embodiment, the enzyme is immobilized by crosslinking mechanism to the nanofibers. In another embodiment of the method, the enzyme is immobilized by covalent reaction to the nanofibers. In one example, the first output is ammonium, the second output is L-Glutamate, and the final output is glutamine.

The invention is further explained in the following examples, which however, are not to be construed to limit the scope of the invention.

### EXAMPLES

### Example—1

Metabolic paths are the most general way for elimination and production of natural materials by nature. Enzymes play key role amongst these methods. However, instability of enzymes is a critical problem ahead. Immobilization on adequate matrix is an effective solution to overcome this problem. In this manner, due to recent developments in nano industries and nano structures with unique properties, utilization of these materials as a immobilization matrix according to their high surface to volume ratio and high mass transfer ability, results in higher stability and improvement in immobilized enzyme activity.

To fabricate an in-vitro metabolic pathway for absorption of Urea and turning it into a valuable Glutamine amino acid product, Urease, Glutamine Dehydrogenase (GDH) and Glutamine Synthetase was immobilized on a cellulose nano-fiber matrix and properties and effects of immobilization on stability of these enzymes were investigated. Enzymes are immobilized on nano-fiber cellulose, which was produced from Acetobacter xylinum, through adsorption and covalent bonds. Afterwards, effects of temperature, pH, stability and reusability are investigated. In the last step, three micro-bioreactor for enzymatic reaction of glutamine amino-acid production are designed and fabricated, and three immobilized enzymes were placed inside micro-bioreactor as a new concept of nano-bioreactor. As a result, extracellular metabolic pathway of urea uptake is simulated and used effectively.

### Example—2 Cellulose Nano-Fiber Synthesis

Acetobacter xylinum bacterial strain is used with PTCC No. 1374 which was supplied from Iran's national scientific and industrial research organization. HS and molasses medium is used and each medium is cultivated along with cultivation media for 7 and 15 days.

TABLE 1
HS medium components

| Substance | w/v % ratio |
|---|---|
| Glucose | 2 |
| Peptone | 0.5 |
| Yeast extract | 0.5 |
| Anhydrous Disodium Phosphate | 0.27 |
| Monohydrate Citric Acid | 0.15 |

TABLE 2
Molasses components (CSL)

| Substance | Quantity |
|---|---|
| Treated molasses | 110 gr/lit |
| Anhydrous Disodium Phosphate | 27 gr/lit |
| Citric Acid | 1.15 gr/lit |
| CSL | 80 ml/lit |

Acetobacter xylinum bacteria is used to all nano-fiber synthesize with different synthesize duration and different carbon source. Nano-fiber A and B are synthesized identically in HS medium and the only difference was synthesis duration which was 7 days for nano-fiber A and 15 days for nano-fiber B. For nano-fibers D and C, molasses medium is used and durations are 7 days and 15 days respectively.

TABLE 3
Synthesized nano fibers

| No. | Medium | Duration (days) | Name |
|---|---|---|---|
| 1 | HS | 7 | (A) |
| 2 | HS | 15 | (B) |
| 3 | Molasses | 7 | (C) |
| 4 | Molasses | 15 | (D) |

Cultivation media condition is: used pH for nano-fiber synthesis is 4.5, medium temperature is 23 to 24 degrees of Celsius, Cultivation media age: cultivation media used for all nano-fibers synthesis is 4 days aged, Medium quantity: cultivation media to medium ratio in all experiments is 7.5%. The coverage of sterilized condition is one of the most important steps of procedure. Insofar as, disrespect to this step can result in reduction or even absence of production. The conditions are as follows, (1) sterilization of media using 70% alcohol, (2) washing of all components with detergents before every cultivation, (3) exposing of all devices and components to UV ray in microbial hood for 6 hours, (4) sterilization of all component with 70% alcohol under hood, (5) continuation of procedure beside flame, (6) at the end of cultivation 0.01% of nystatin is added to medium, where nystatin prevents any kind of fungus growth and contamination in medium. After synthesis of medium in optimum condition of pH=4.5 and addition of cultivation media with ratio of 10%, vessel is sealed thoroughly and coated with parafilm. During 7 day cultivation, vessel is not opened in any condition and is kept in room temperature.

### Example—3 Immobilization of Urease

The urease solution (1 to 5 mg mL⁻¹) was prepared in phosphate buffer (pH=6.0 to 9.0, concentration of 10 to 100 mM). 1 ml of the urease solution was dropped on bacterial cellulose nanofiber sheet, it remained for 3 h at room temperature (25° C.). Then the glutaraldehyde solution with concentration of 0.5 to 2% w/v was added to the urease solution on the mentioned nanofiber sheet for 17 hours. Afterwards, for finalizing the immobilization, the bacterial cellulose nanofiber sheet was washed by phosphate buffer and vortexed for about 10 min. Urease activity for free and immobilized enzymes were determined by the Berthelot method. One unit of enzyme activity was defined as the amount of enzyme which can hydrolyze 1 μmol urea per minute at 37° C. under the assay conditions.

In order to determine the reusability of the immobilized urease, its activity was measured by using the same composite cellulose nanofiber for ten times in urea hydrolysis. All the experiments were conducted at room temperature with the same conditions of reaction. For determining the stability of Free and immobilized ureases were stored at laboratory temperature. Samples were taken each week and first day activity was set to 100%.

### Example—4 Effect of pH on the Immobilized Urease

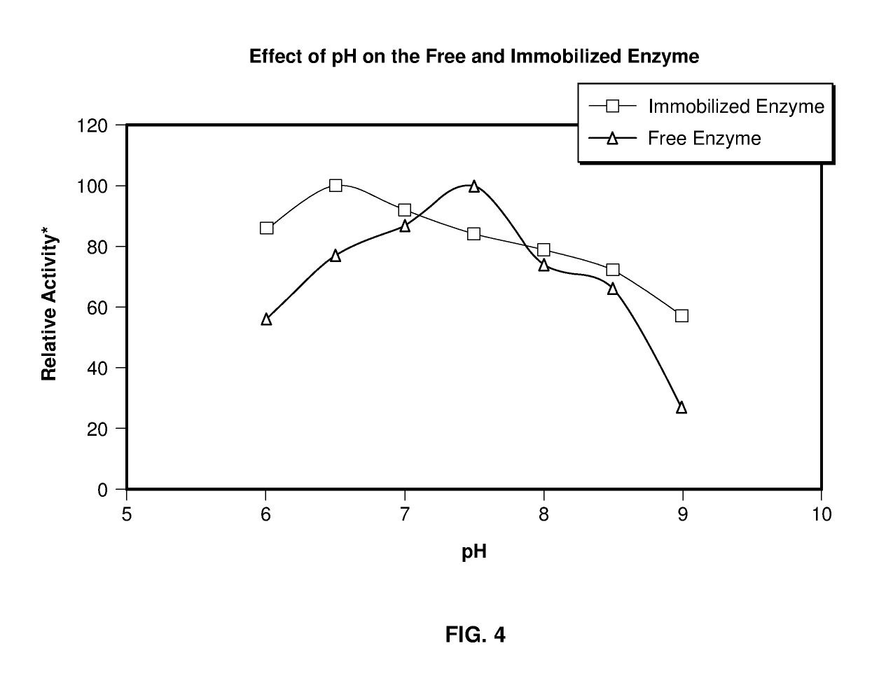

Effect of pH on the free and immobilized enzyme activity was studied. As it illustrated in FIG. 4, the obtained results showed that the optimized pH for the free enzyme was 7.5 but this value for the immobilized enzyme was 6.5. The reason for the decrease of the proper pH for enzyme activity in the immobilized form, can be interpreted on the basis of the effect of diffusional effects in the cellulosic nano-fibers. When the urease enzyme is active, the product is ammonium which is faced to a resistance of the nano-fiber during its exit from the fiber. This resistance caused the difference between pH of the bulk and its value in the pores which is the reason of difference in the optimized environmental pH for the free and immobilized urease. The relative activities at the optimum pH were taken as 100% for free and immobilized Urease, respectively.

### Example—5 Effect of Temperature on the Immobilized Urease

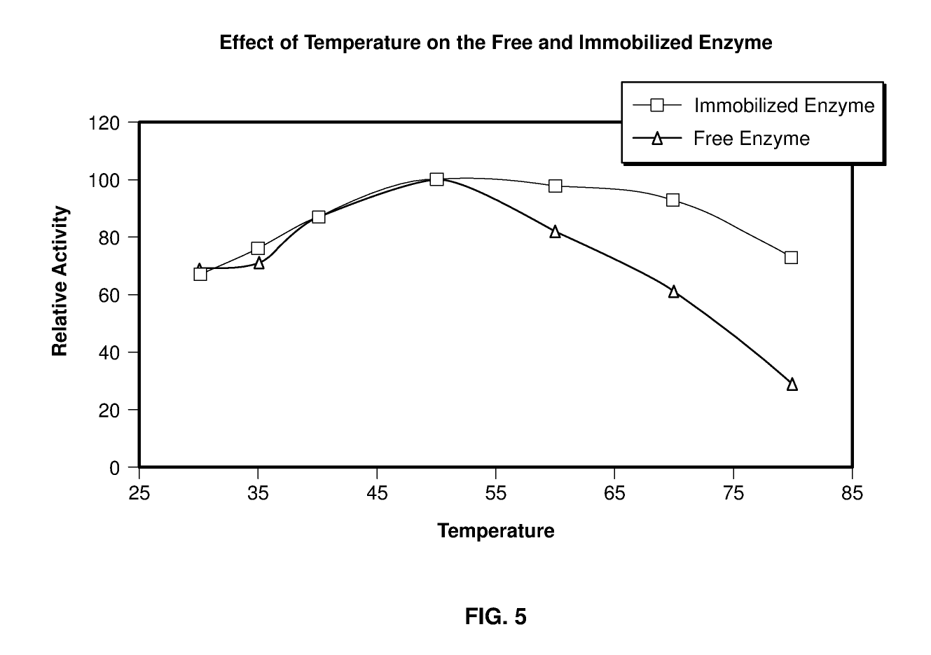

Effect of temperature on the free and immobilized enzyme activity was studied in the range of 30° C. to 80° C. As it is shown in FIG. 5, the optimized temperature for both free and immobilized enzyme was 50° C., but the sensitivity of the immobilized enzyme to varying temperature was less than the free enzyme, especially in the higher ranges of temperature which may cause enzyme denaturation. It should be noted here that the nano-pores immobilized enzyme, which is linked to each other by glutaraldehyde, caused some spatial limitation on the conformational changes of the enzyme and this causes better performance of the immobilized enzyme in comparison to the free enzyme in higher temperature.

Crosslinking of enzymes to each other and formation of enzymatic network decrease the possibility of their conformational change at temperature higher than the optimum one. For this reason, the enzyme retain its activity at temperature above 50° C. and its activity decreased only about 30% at 80° C. while this decline was about 94% for the free enzyme. The stability of enzyme against denaturation above the optimum temperature was reported and summarized in Table 4, the activity maintenance in the present work was higher comparing to the previous studies. The reason can be immobilization of the urease in the nano space of the supports used and showed that the resistance of urease against conformational changes increases because of immobilization and cross linking.

TABLE 4
Comparing the effect of temperature increasing on immobilized urease
activity

| | | | | Enzyme Activity in | | |
|---|---|---|---|---|---|---|
| | Immobilization | Method of | Optimum | Opt Tem + X ° C. | | |
| Enzyme | Matrix | Immobilization | Temperature | 10° C. | 20° C. | 30° C. |
| Urease | Bacterial | Cross linking | 50° C. | 98 | 93 | 73 |
| | Cellulose | | | | | |
| | Nanofiber | | | | | |
| Urease | Electrospun | Covalent | 50° C. | 60 | 45 | 30 |
| | polyacrylonitrile | Bonding | | | | |
| Urease | Nanostructured | Covalent | 30° C. | 90 | — | — |
| | polymer | Bonding | | | | |
| Urease | Sodium | Cross linking | 50° C. | 60 | 30 | — |
| | Alginate | | | | | |
| Urease | Cellulosic | Covalent | 35° C. | 75 | — | — |
| | cotton fibers | Bonding | | | | |

### Example—6 Urease Stability and Reusability

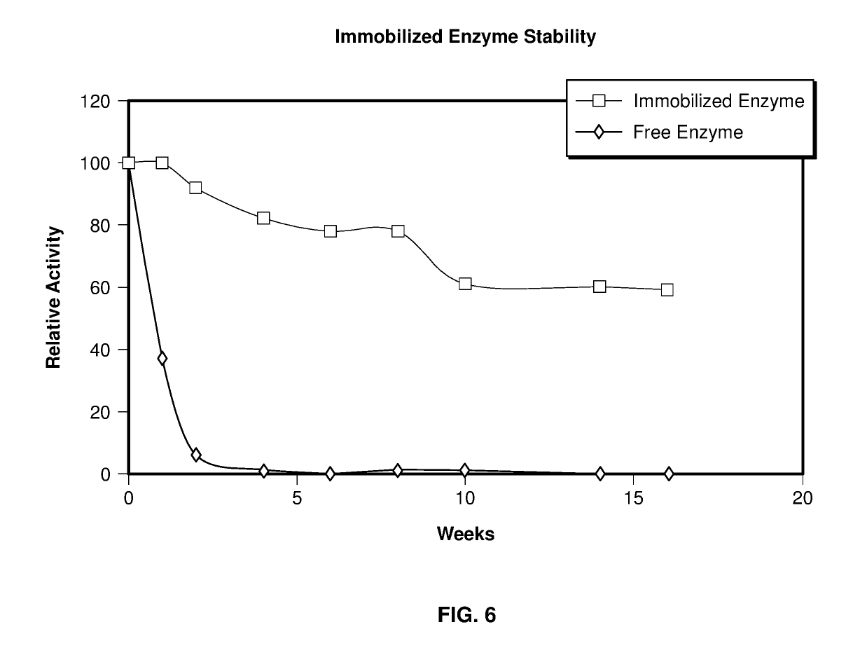

One of the main advantages of enzyme immobilization is the possibility of its multiple use in the bioprocesses and the other one is their stability during a long period of time. FIG. 6 shows that using the mentioned enzyme after 14 days, only 8% decrease in the enzyme activity was seen and after 120 days, this decrease was only 41%. It should be mentioned here that the free enzyme lost about 73% of its activity after 7 days in our laboratory condition (28° C.).

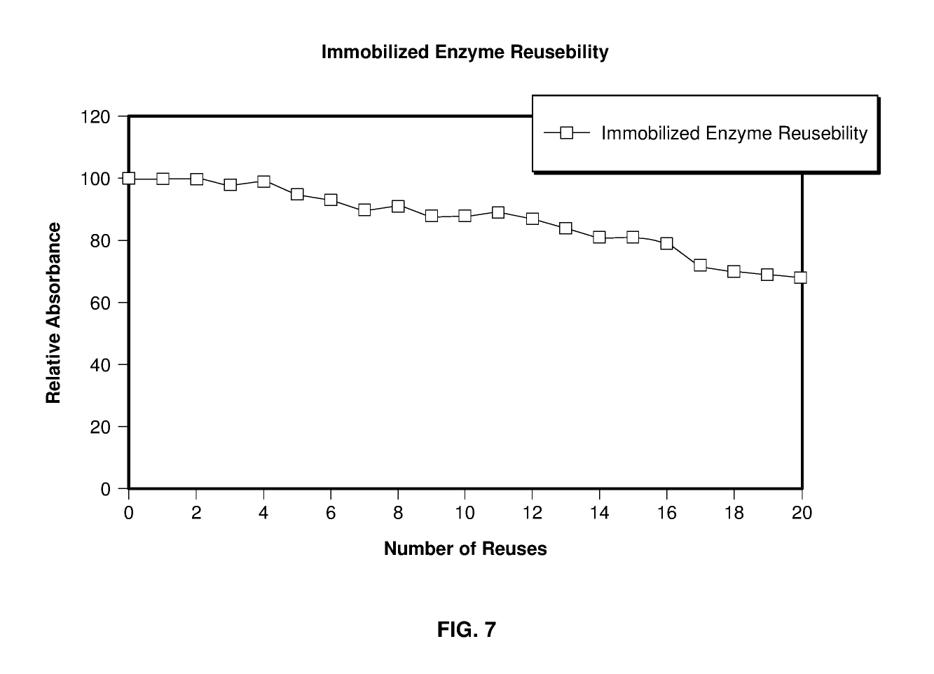

FIG. 7 demonstrate that, the immobilized urease activity decreased only slowly after it was measured repeatedly. The enzyme retained more than 87% of its initial activity after 10 times of reuse cycles. Also, this system maintained around 68% of its initial activity even after 20 cycles. Normally, immobilization inhibits enzyme to lose its 3-dimensional conformation and active site and also its activity during repeated number of using. Besides, in this study because of the crosslinking of urease in nanofiber structure strong enzyme aggregates was formed which can stop the enzyme leakage in the solution and also prevents its denaturing during different cycles used.

### Example—7 Immobilization of Glutamate Dehydrogenase

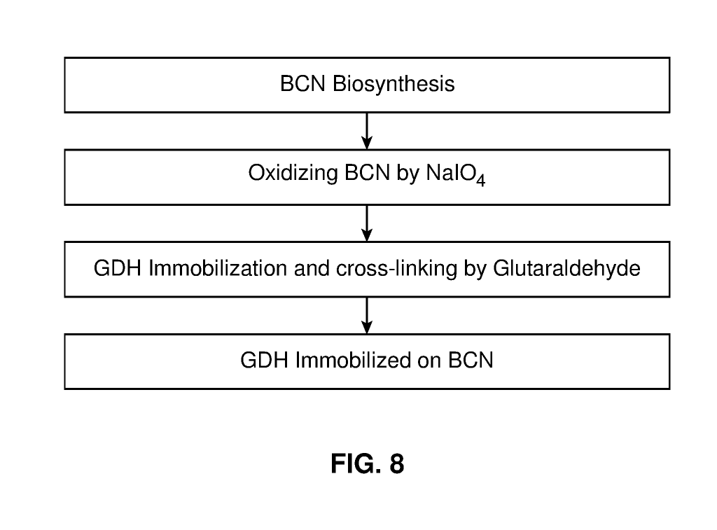

The glutamate dehydrogenase solution (2 mg mL⁻¹) was prepared in phosphate buffer (pH=7, concentration of 30 mM). 2 mlmg⁻¹ of the glutamate dehydrogenase solution was dropped on BCN sheet, remained for 3 h at room temperature (25° C.). Subsequently the glutaraldehyde solution with concentration of 0.75% w/v was added to the glutamate dehydrogenase solution on the mentioned nanofiber sheet for 17 h. To finalize the immobilization, the BCN sheet was washed by phosphate buffer and vortexed for about 10 min. Immobilization process is demonstrated in FIG. 8.

GDH catalyzes the reaction:

α-Ketoglutarate+NH₄⁺++ATP+NADPH->L-Glutamate+NADP+ADP+Pi

The enzyme was assayed spectrophotometrically using the reverse reaction, by monitoring the decrease in absorbance at 340 nm due to NADH. The enzyme reusability was measured during 10 times reusing. All the experiments were conducted at room temperature with the same conditions of reaction. To determine the stability of free and immobilized glutamate dehydrogenase, they were stored for 56 days at laboratory temperature. Samples were taken weekly. The first day activity was considered as 100%.

### Example—8 Determination of the Protein Content of Immobilized Glutamate Dehydrogenase Nanofibers

The determination of protein content using Bradford method revealed that BCN after immobilization of GDH, contains 1.84 mg ml-1 of protein initially. Determination of protein content after 10 times reusing and stability test during the 8 weeks indicates enzyme immigration from BCN.

The CNF (carbon nanofibers) surface consists of mainly —OH functional group that can be directly interacted weakly with enzyme, and its binding can be improved by surface modification and interaction of chemical coupling that forms a strong and stable covalent immobilization of enzyme. The covalent interaction for enzyme immobilization is important to provide more efficient interaction between CNF support and enzyme molecule. Enzyme immobilization onto CNF is having potential for improving enzymatic performance and production yield, as well as contributing toward green technology and sustainable sources.

### Example—9 Effect of pH on Immobilized Glutamate Dehydrogenase

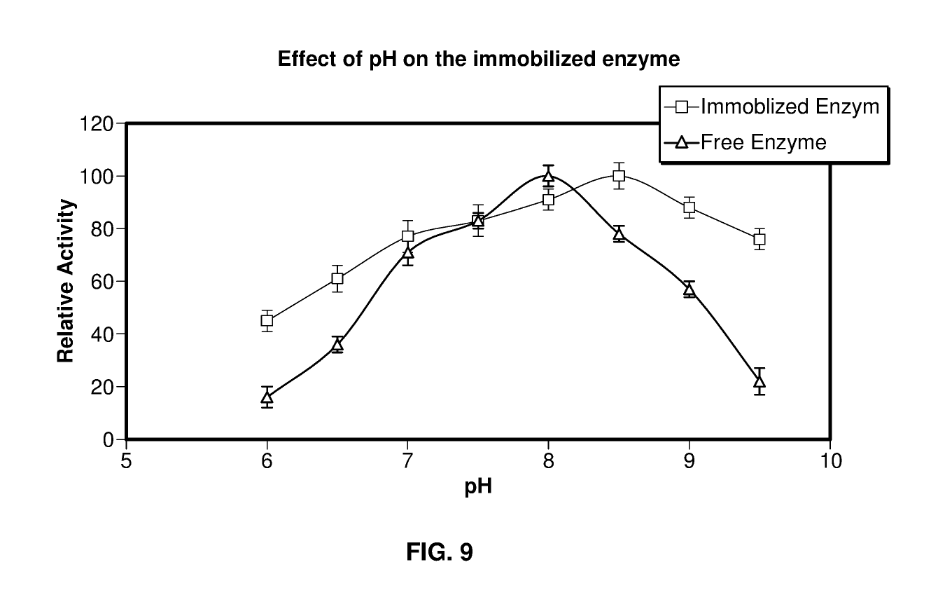

The effect of pH variation on the free and immobilized enzyme activity have been investigated as shown in FIG. 9. This effect was examined in the range of 6.0 to 9.5. The obtained results showed that the optimized pH for the free enzyme was 8. The increase of the proper pH for enzyme activity in the immobilized form, can be interpreted based on diffusional effects in the cellulosic nanofibers. The active glutamate dehydrogenase enzyme consuming the specific reactant (NH₄⁺) during its transfer from the nanofiber which is facing with the resistance of the nanofiber. This resistance causes the difference between pH of the bulk and its value in the pores. This result is expected because the GDH uses NH₄⁺ as substrate which would make the local nano-environment around the bound GDH more acidic, and also due to mass transfer resistance between BCN and environment optimum pH for bulk would be shifted to higher values.

In the most of the studies which have used the macro structures as immobilization supports, the results show an increase in optimum pH comparison to the optimum activity range of free enzyme. The reason behind differing pH is the different reaction direction. GDH is an allosteric enzyme and it can catalyze reverse reaction, and gives completely different optimum pH for free and immobilized enzyme.

The enzyme GDH was also able to catalyze the inverse reductive reaction. At acidic pH values, the dissociation of enzyme subunits produced the rapid enzyme inactivation even at 25° C. It was reported that the multimeric structure of the enzyme was stabilized by the immobilization (enzyme subunits could not be desorbed from the support by boiling it in the presence of sodium dodecyl sulphate). This makes the enzyme stable at various pH and even improved the enzyme stability at neutral pH values. This immobilized enzyme can be of great interest as a biosensor or as a biocatalyst to regenerate both reduced and oxidized cofactors.

Enzyme immobilized biochemical sensors represent an important subclass of biosensors. Among these, a glutamate dehydrogenase (GDH) based biosensor could have important applications. GDH reversibly catalyzes the oxidative deamination of glutamate to ketoglutarate and ammonia using NAD+/NADH or NADP+/NADPH as coenzymes. Glutamate is a key intermediary in the transfer of amino groups between different amino acids, and therefore in amino acid synthesis and degradation.

### Example—10 Effect of Temperature on the Immobilized Glutamate Dehydrogenase

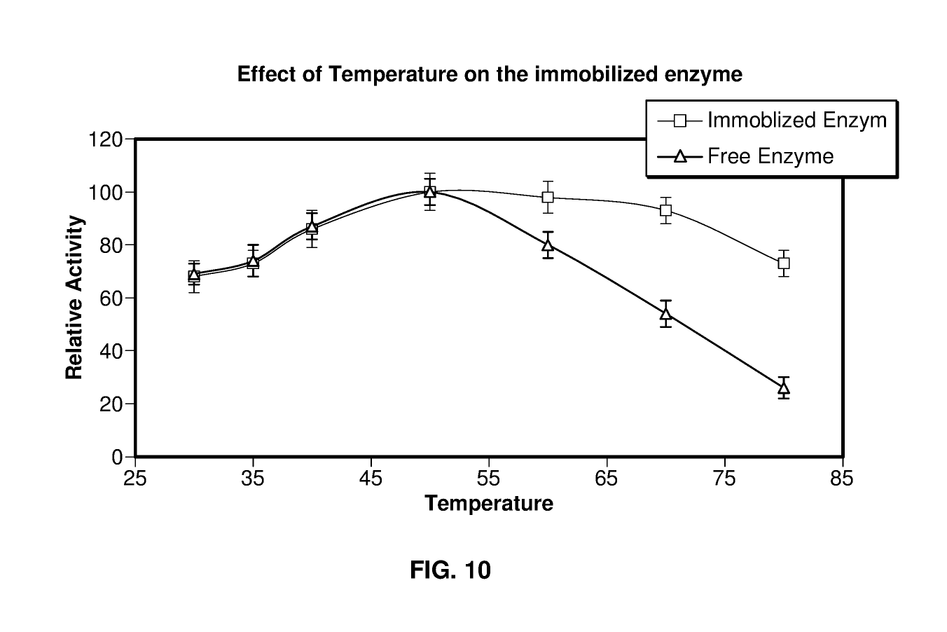

The effect of temperature on the free and immobilized enzyme activity has been studied in the range of 30 to 80° C. (FIG. 10). The optimal temperature for both free and immobilized enzyme was around 50° C. but the sensitivity of the immobilized enzyme to temperature variations was less than the free enzyme in all cases, especially in higher temperature which may cause the enzyme denaturation. It should be noted that the immobilized GDH in nonporous, which are linked to each other and the matrix by gluteraldehyde, are the sources of some spatial limitation on the conformational changes of the enzyme. At the same time, it results in better performance of the immobilized enzyme in comparison to the free enzyme in higher temperature.

As it is demonstrated in FIG. 10, free and immobilized enzyme have almost the same trend before optimum temperature (50° C.), while in higher temperature, the synthesized BCNs shows better performance for immobilized enzyme. In lower temperature, conformational changes does not occur, also the relative activity of immobilized and free enzyme shows the same trends. However, in higher temperature conformational changes was observed in free enzyme, therefore the activity of the free enzyme decreased because of denaturation phenomena.

Crosslinking of enzymes to each other and formation of enzymatic network decreases the possibility of their conformational change at temperature above than the optimum. In consequence, the enzyme almost retains its activity at temperature above 500° C. and decreases just about 21% at 800° C. while the reduction is about 94% for the free enzyme. Effect of temperature on immobilized GDH has not been reported in any prior known arts.

### Example—11 Study on the Glutamate Dehydrogenase Stability

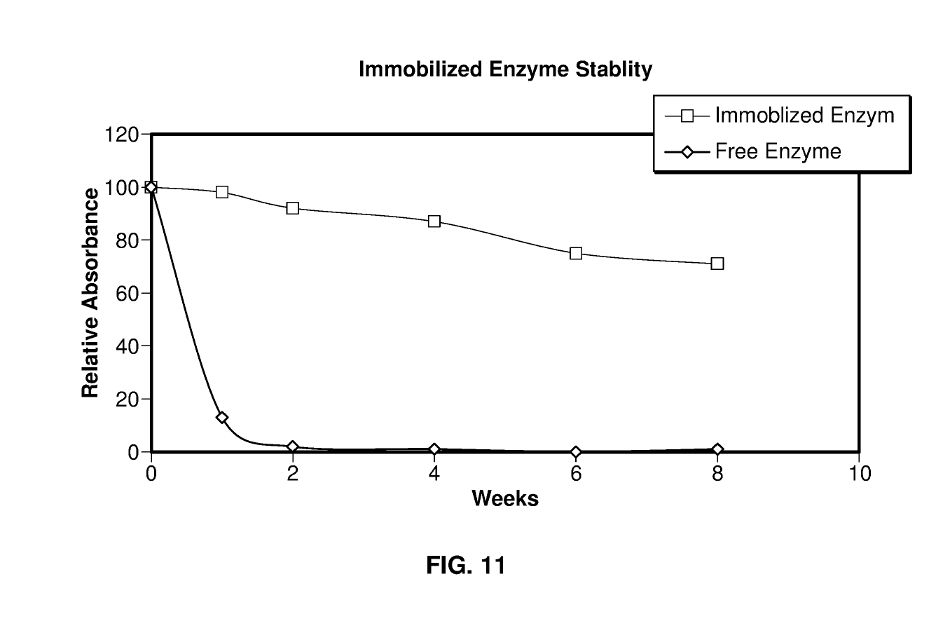

One of the main advantages of enzyme immobilization is the possibility of its multiple use in the bioprocesses and the other one is their stability during a long period of time as shown in FIG. 11. With using the GDH immobilized after 8 weeks, the enzyme activity decreased only 36%, whereas protein content decrease to 1.59 mg·ml⁻¹ from initial protein of 1.84 mg·ml⁻¹. It should be mentioned that, the free enzyme lost around 90% of its activity after 2 weeks in our laboratory condition (28° C.). Like temperature, stability of immobilized GDH has not been reported in in any prior known arts.

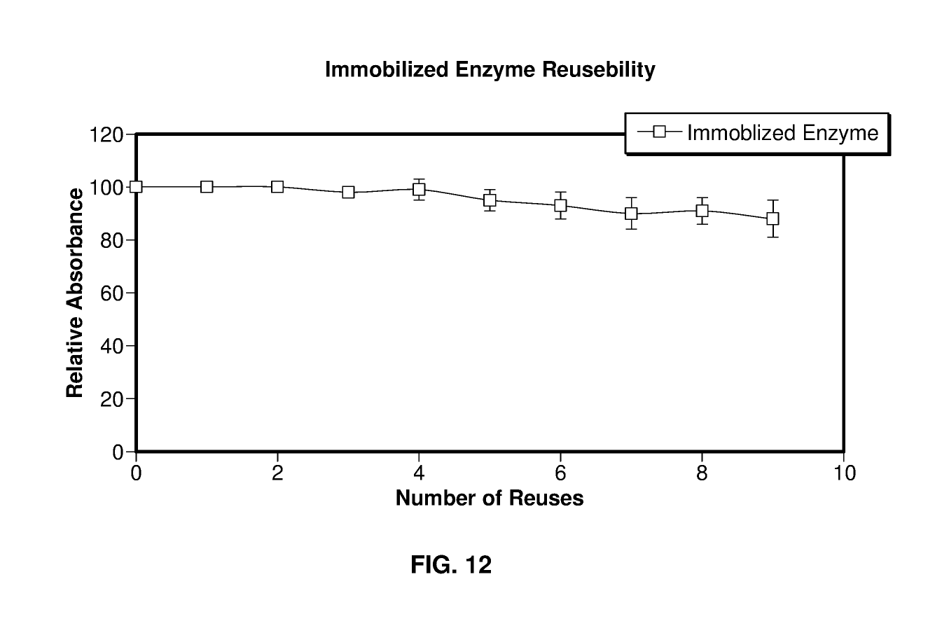

Enzyme reusability is one of the main advantages of immobilization. The immobilized GDH activity decreased only slowly after it was reused repeatedly as shown in FIG. 12. The enzyme retained more than 83% of its initial activity after 10 times of reuse cycles, whereas protein content decreases to 1.78 mg·ml⁻¹ from initial protein of 1.84 mg·ml⁻¹. Normally, immobilization inhibits enzyme to lose its three-dimensional conformation and active site and also its activity during repeated number of using. Besides, because of the crosslinking of GDH in nanofiber structure strong enzyme aggregates were formed which can stop the enzyme leakage in the solution and also prevents its denaturing during different cycles used.

### Example—12 Kinetic Studies of Glutamate Dehydrogenase

The correlation (Lineweaver-Burk plot) between the conversion rate of substrate by enzymatic reaction and substrate concentration provide information about the kinetic parameters i.e., Vₘ (maximum reaction rate) and Kₘ (the Michaelis constant). Kₘ shows the enzyme affinity to the substrate and Vₘ, demonstrates the maximum velocity that can be theoretically reach in the enzymatic reaction. For the free and immobilized GDH Kₘ were 0.037 mM and 0.11 mM respectively. Moreover, the Vₘ for the immobilized and free GDH were 4.06 (μmol NADPH. min⁻¹·mg immobilized GDH⁻¹) and 6.16 (μmol NADPH. min⁻¹·mg free GDH⁻¹) respectively. Like the other immobilizations, affinity of GDH to substrate decreases and resulted in an increase in Kₘ for GDH immobilization. Ineffective substrate access to the immobilized enzyme active site result in the increase of Kₘ that cause the lower rate of the reaction and Vₘ.

The most important causes that clarify the reason for difficult reaching of the substrate to the enzyme active site after its immobilization on a solid support are as follows: mass transfer limitations within the support structure, conformational changes of enzyme during the immobilization and washing steps, restricted freedom of movement due to multipoint crosslinking, and non-bio-specific orientation of the enzyme active site through the bacterial cellulosic nanofiber.

### Example—13 Immobilization of Glutamine Synthetase

All reactions associated with glutamate dehydrogenize activity assessment was conducted using CAK1021 kits from Cohesion Bioscience company. Glutamine synthetase activity assay in based on gamma-glutamine transferase activity of GS and is assayed by gamma-glutamyl hydroxamate. Quantity of the produced gamma-glutamyl hydroxamate in supernatant is estimated with measurement of absorption at 540 nm. As mentioned in previous sections, cellulose nanofibers were used in this study.

According to the data provided in protein data bank and in some references, size weight of glutamine synthetase is 450 Kilo Dalton. The images obtained from SEM, this enzyme shall not be observable. However, immobilized enzyme of glutamine synthetase with crosslinking to matrix and to each other can be observed as an almost smooth tissue on nanofiber. Furthermore, this image shows that the immobilized glutamine synthetase does not generate a detached structure from nano fiber and after immobilizing glutamine synthetase on nanofiber surface, an organized and homogenous structures is obtained which is used as immobilized glutamine synthetase in experiments.

### Example—14 Determination Protein Content for Immobilized Glutamine Synthetase Enzyme

To determine protein content inside nanofiber for glutamine synthetase enzyme, Bradford method has been used. Different protein content inside nanofibers were determined and measured using this method. Protein content in cellulose nanofibers from medium D was 0.8 mg/ml. It should be mentioned that stability and reusability of enzymes on nanofibers are independent of immobilized protein inside tissue and nano structure since proteins can be active or inactive which will be determined in various upcoming experiments. However, absorbed protein inside a nano structure is interdependent with structure capacity in holding and implanting enzyme inside its structure. Bradford method solely determines enzyme content in nano structures which includes both active and inactive parts of enzyme. Activity of free and immobilized glutamine synthetase enzyme is presented in Table 5. Activity of immobilized glutamine synthetase enzyme is obtained based on amount of immobilized protein from previous section.

TABLE 5
Effect of immobilization matrix on immobilized glutamine synthetase
activity

| | | | Glutamine |
|---|---|---|---|
| | | | Synthetase Activity |
| Experiment | | Immobilization | μ · mol |
| number | Enzyme | Matrix | GSmin−1mg−1 |
| — | Glutamine | free | 4.93 |
| | Synthetase | | |
| 1 | Glutamine | Bacterial Cellulose | 2.19 |
| | Synthetase | Nanofiber | |

### Example—15 Assessment of Free and Immobilized Glutamine Synthetase: Performance on Cellulose Nanofiber in Various pH

Effect of pH on free glutamine synthetase was studied. As it can be seen in FIG. 11, effect of pH in range of 6-9.5 was assessed on glutamine synthetase enzyme and results showed that the optimum pH for this experiment was 7.5 for free enzyme. Unlike urease and glutamate dehydrogenize; optimum pH does not change for immobilized enzyme. The main difference lies on the performance of free and immobilized enzyme.

On the other hand, there are few articles and researches on this topic in literature comparing to other two enzymes. For this reason, all experiments conducted in this section will not be comparable with other articles and papers, there for shall be compared with glutamate dehydrogenase and urease with approximately same condition. It should be noted that achieved optimum pH in this study is compatible with other researches and has a good agreement with other researchers results. It is reported that, optimum pH of 7 for some sorts of glutamine synthetase and 7.5 for purified glutamine synthetase.

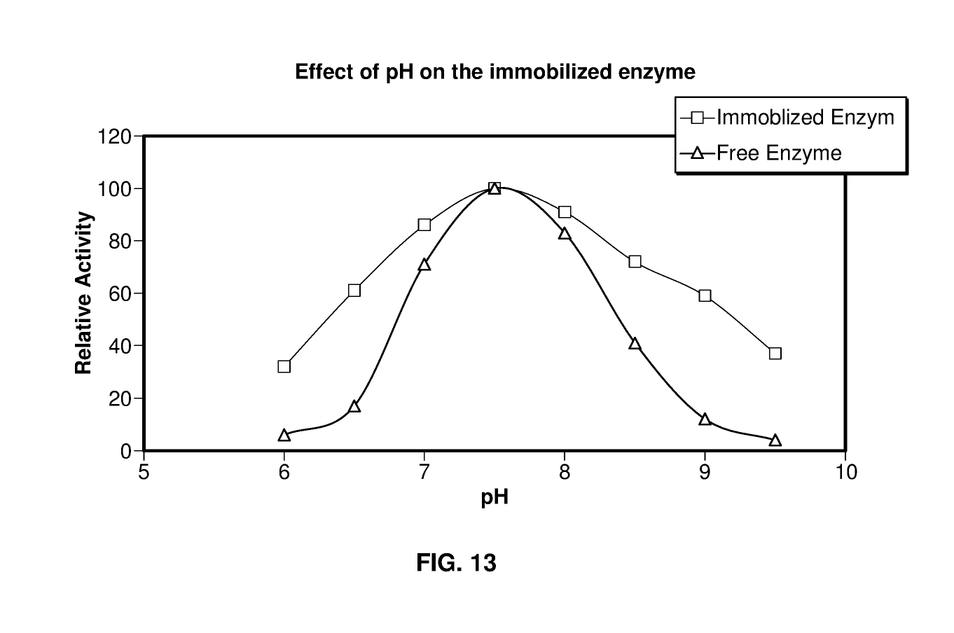

With respect to FIG. 13, it can be seen that immobilization of glutamine synthetase on cellulose nanofiber results in better performance and increased efficiency of enzyme in higher range of differing pH. Glutamine synthetase is less stable against pH variation in comparison with other two enzymes in this study. This enzyme loses its activity up to 88% in 1.5 unit of pH variation form optimum pH. Whereas activity lose in this range for urease and glutamate dehydrogenase was about 70%.

After immobilization of glutamine synthetase, enzyme performance in pHs other than optimum pH was improved. As it can be seen in FIG. 13, 61% of enzyme activity in process was maintained in pH of 9. In pHs of more acidic than optimum pH, enzyme activity was maintained and in pH of 6 only 68% of enzyme activity was lost whilst free enzyme activity at this pH was only 6%.

### Example—16 Effect of Temperature on Free and Immobilized Glutamine Synthetase

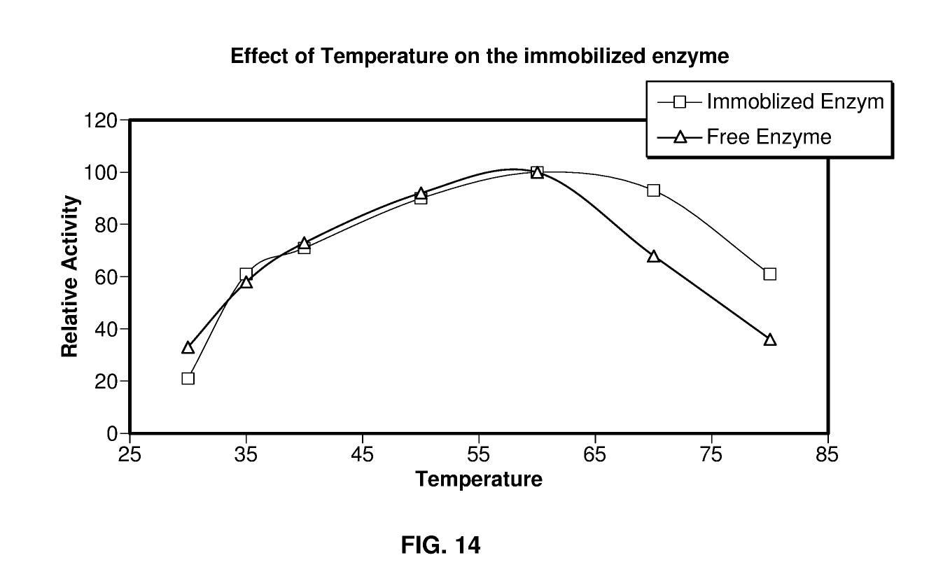

Free and immobilized glutamine synthetase were examined in temperature ranging from 30 to 80 degrees of Celsius. As it can be seen in FIG. 14, optimum temperature for free and immobilized enzyme lies in range of 60 degrees of Celsius. However, immobilized enzyme shows less sensitivity to temperature variation in comparison to free enzyme and this subject has been mentioned in pervious articles as well and one of enzyme immobilization reasons on nano structures is spatial limitation on the conformational changes in enzymes. As for, immobilized enzyme shows less sensitivity in higher temperature and immobilization prevents enzyme denaturation and helps maintenance of enzyme activity. It worth mentioning that there are few studies on glutamine synthetase and not much is available to compare with results and performance review of immobilized glutamine synthetase enzyme is one of important innovations of this study.

As presented in FIG. 14, behavior of examined immobilized glutamine synthetase on cellulose nanofibers shows a specific procedure. However, results show that, immobilized glutamine synthetase on cellulose nanofiber is less stable to pH and temperature variation in comparison with urease and glutamate dehydrogenase. As it can be noted in FIG. 14, crosslinking for examined glutamine synthetase provides an integrated enzyme and matrix which results in relatively resistant glutamine synthetase in higher temperature that leads to only a 39% activity loss in temperature higher than 60 degrees of Celsius (optimum temperature) up to 80 degrees of Celsius whereas this percentage exceed 64% for free enzyme. It is needless to mention that these parameters show better results in glutamate dehydrogenase and urease. Immobilized urease maintain its activity by 84% in 80 degrees of Celsius and that is 73% for glutamate dehydrogenase.

### Example—17 Free and Immobilized Glutamine Synthetase Stability

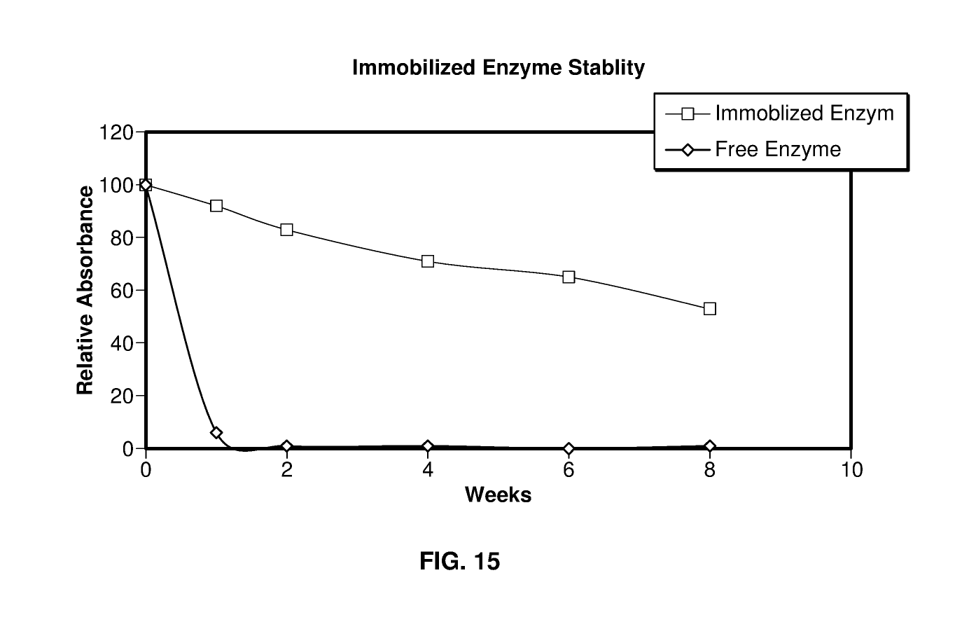

Stability of glutamine synthetase enzyme was studied. Another advantage of immobilized enzyme comparing to free enzyme is stability. Long-term activity loss experiment for cellulose nanofiber was conducted and as it can be seen in FIG. 15, results show a 17% activity loss during 14 days and 47% activity loss during 56 days for immobilized glutamine synthetase on cellulose nanofiber.

Results presented in this section of studies cannot be found in literature. Nevertheless, in comparison to urease and glutamate dehydrogenase, they showed a better performance on this matter as well which was 78% activity maintenance and 71% activity maintenance after 8 weeks respectively.

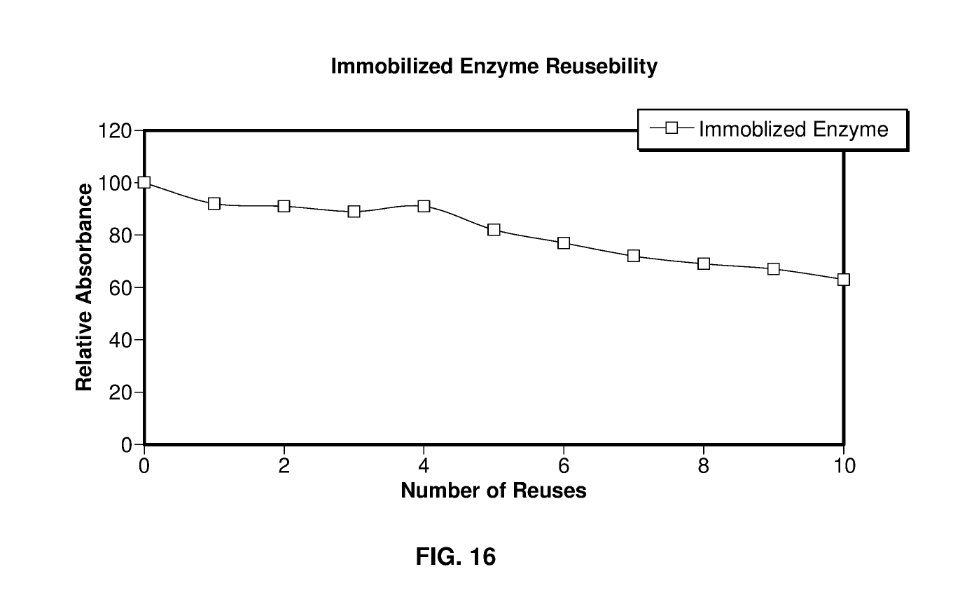

Another results implying improved performance of immobilized enzyme is reusability experiment. This experiment was conducted on immobilized glutamine synthetase on cellulose nanofiber. As shown in FIG. 16, immobilized glutamine synthetase on cellulose nanofiber which was synthetased in D medium, shows an acceptable performance in this survey. Immobilized enzymes maintained their activity by 83% after 5 cycles of use. As it can be seen in FIG. 16, immobilized enzyme maintained its activity by 64% after 10 cycles of use.

Spatial limitation on conformational change and preserving enzyme active site caused by enzyme immobilization and it increased the reusability of enzyme. It should be mentioned that in this study, feasibility of immobilized glutamine synthetase utilization was less in comparison to other enzymes due to higher price of this enzyme. Therefore, further studies on this enzyme seems essential. As to knowledge of writer, no result has been reported on immobilized glutamine synthetase.

### Example—18 Kinetic Constants Determination for Immobilized Glutamine Synthetase on Cellulose Nanofiber

In this stage, kinetic constants for immobilized glutamine synthetase on cellulose nanofiber matrix have been studied. In this stage, Lineweaver-Bulk method has been used to measure kinetic constants and Vm parameter showing maximum reaction rate and Km parameter as Mikailsen Menten constant was utilized for assessment of this matter. Km Shows affinity of enzyme to substrate and Vm is the maximum theoretical velocity which can happen in an enzyme. For free enzyme, Km is 1.9 mM and for immobilized enzyme it is 2.87 mM. Vm for free and immobilized enzyme is 0.011 mMol/min and 0.003 mMol/min respectively.

For this purpose, procedures conducted on immobilized enzyme activity assay were executed continuously and after each use, immobilized enzyme was washed with phosphate buffer and reused again. During this assay, temperature is kept at room temperature and identical condition is provided for each reuse.

### Example—19 Micro-Bioreactor Assembly

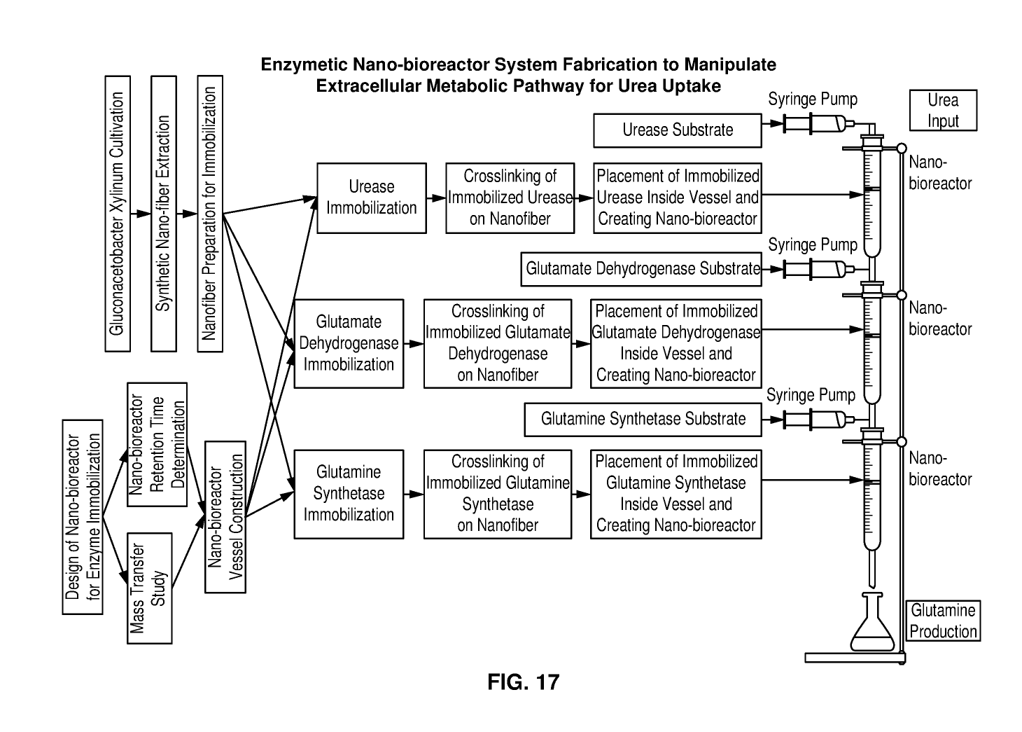

To conduct experiments, 3 micro-bioreactors are designed and fabricated with glass and jointed with silicon connector. Dimensions of micro-bioreactor are illustrated in Table 6. Moreover, schematic shape of aforementioned micro-bioreactor is shown in FIG. 17. As shown in this figure, reactors are injected via syringe pump into container. Inside container, nano-fiber matrix containing immobilized enzyme is placed on a supporter. After entering inside container and passing through nanofibers containing immobilized enzymes, substrates begin to react and final product is extracted from bottom of the container.

All mass transfer and hydrodynamic simulation's data is presented in corresponding section to obtain engineering parameters of assembled micro-bioreactor, flow condition inside micro-bioreactor is retrieved using hydrodynamic relations and ComSol software.

Configuration of micro-bioreactor is achieved using micro-bioreactor internal flow simulation with several drafting in ComSol software. This part of the project is conducted in research center of new technologies of life science engineering research center (INFS) of Tehran University under supervision of Dr. Amouabedini.

TABLE 6
Operative specifications of container

| Height | 3 cm |
|---|---|
| Diameter | 1 cm |
| Temperature | 40° C. |
| pH | 8.5 |

### Example—20 Extracellular Urea Absorption Metabolic Pathway

After choosing appropriate immobilization matrix for Urease, Glutamate dehydrogenize and Glutamine Synthetase on cellulose nano-fiber, manipulating of extracellular urea metabolic pathway was reachable. According to definitions, the term Nano-bioreactor is used for a nano-space inside which a controlled bio-chemical reaction is feasible. Given this definition, enzyme nano-particles, immobilized enzymes on nano-fibers and immobilized enzymes and proteins on mesoporous can be classified as nano-bioreactors. Nano-bioreactor assembly needs container for which a micro-bioreactor with operative volume of 200 micro liter is provided. Immobilized enzymes in nano-fibers are placed inside this micro-bioreactor.

According to values obtained in kinetic constant section, retention time for each nano-bioreactor is calculated and based on maximum specific velocity in Table 7, limiting reaction is selected and all corresponding flow rates, substrate flows and concentrations are calculated.

TABLE 7
Maximum specific velocity of immobilized enzyme activity on cellulose
nano-fiber matrix

| No. | Enzyme | vmax |
|---|---|---|
| 1 | Urease | mMol/min/mg 3.1 |
| 2 | Glutamate | 0.406 μMol NADPH/min/mg |
| | Dehydrogenized | |
| 3 | Glutamine Synthetase | 0.003 mMol/min/mg |

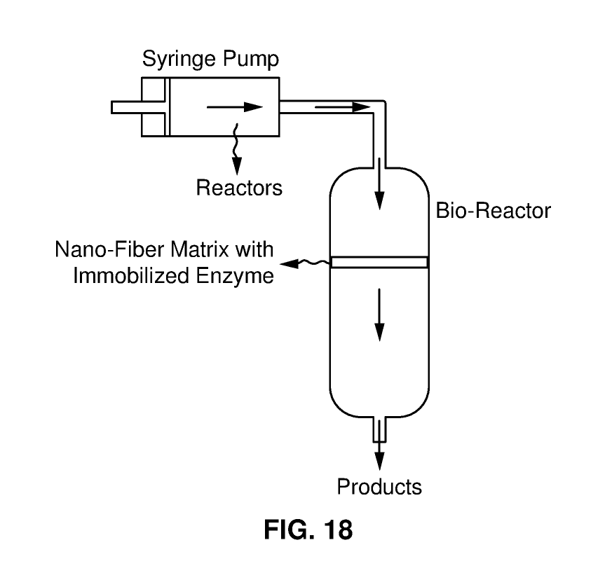

Thermal condition for extracellular metabolic pathway experiment is kept at 25 degrees of Celsius and for all experiments pH is 7. Schematic outline of fabricated extracellular metabolic pathway is presented in FIG. 18. After conduction early experiments, conversion percentage in each section is given in Table 8.

TABLE 8
Maximum specific velocity of immobilized enzyme activity on cellulose
nano-fiber matrix

| | | Conversion percentage with respect |
|---|---|---|
| No. | Enzyme | to single enzyme condition |
| 1 | Urease | 94 |
| 2 | Glutamate | 61 |
| | Dehydrogenized | |
| 3 | Glutamine Synthetase | 57 |

Constructed system is kept out of light and in room temperature for 4 weeks and then reused again. Results obtained from reaction conversion percentage is presented in Table 9.

TABLE 9
Maximum specific velocity immobilized enzyme activity on cellulose
nano-fiber matrix

| | | Conversion percentage with respect to |
|---|---|---|
| No. | Enzyme | single enzyme condition |
| 1 | Urease | 86 |
| 2 | Glutamate | 53 |
| | Dehydrogenized | |
| 3 | Glutamine Synthetase | 47 |

According to results obtained from experiments, it can be concluded that the synthesized extracellular metabolic pathway of urea uptake is constructed which is first steps toward designing and construction of artificial kidney.

### Example—21

At first, two nano-fibers of cellulose and dephenyl-alanine was used for immobilization of urease enzyme. Immobilization condition for urease was optimized. Optimized condition for urease immobilization was buffer concentration of 30 mM, pH=7, Glutaraldehyde concentration of 0.75% (mass to volume) and urease concentration of 2 mg/ml. After immobilization of Urease, optimized performance condition was pH=6.5 and temperature of 50 degrees of Celsius. Immobilized urease lost its activity by 42% after 110 days and after 20 cycles of use 68% of its primary activity was maintained. After Urease, Glutamate dehydrogenized was assessed. At first, immobilization of glutamate dehydrogenized on cellulose nano-fiber was optimized using Genetic Algorithm and thermal condition of 25 degrees of Celsius and pH=7 was obtained as optimized condition for immobilization afterwards.

After immobilization of enzyme, enzyme's activity and performance was surveyed in different temperature. Optimized temperature of performance was assessed as 50 degrees of Celsius like urease and optimized performance pH for immobilized glutamate dehydrogenize was 8.5. Immobilized glutamate dehydrogenizes lost its activity by 29% after 56 days and after 10 cycles of use, 88% of its primary activity was maintained. Synthetic constants of glutamate dehydrogenize was measured and compared with free enzyme. km and Vmax for immobilized glutamate dehydrogenize was 0.11 mM and 0.46 μMol NADPH min/lmg respectively.

After glutamate dehydrogenize, glutamine synthetase was immobilized on cellulose nano-fiber. Optimized performance pH for glutamine synthetase was 7.5 and optimized performance temperature was 60 degrees of Celsius. Immobilized glutamine synthetase lost its activity by 22% after 56 days and maintained its activity by 67% after 10 cycles of reusability. Synthetic constants were obtained and compared with free enzyme. km and Vmax for immobilized glutamine synthetase was 2.87 mM and 0.46 mMol/min respectively.

After immobilization of urease, glutamate dehydrogenize and glutamine synthetase and after surveying their performance separately, they were placed inside a 200 micro-liter of micro-bioreactors. Micro-bioreactors were designed using ComSol software and were fabricated with glass based on hydrodynamic results. Retention time inside micro-bioreactors was obtained based on kinetic constants and volumetric velocities of substrate was adjusted according to retention time. Conversion percentage of each enzyme was surveyed and calculated separately. This percentage was 86% for urease, 53% for glutamate dehydrogenize and 47% for glutamine synthetase. Using ComSol software, optimized flow rate was simulated and flow hydrodynamic was obtained for various velocities. Moreover, substrate and products concentration variations and Reynolds variation inside container was studied. With studies and experiments, extracellular urease uptake pathway with 3 immobilized enzymes on synthetased nanofiber matrix was established and hydrodynamic and mass transfer models were generated.

### Example—22

Modulation of bio-reactors at nano scale with bio-catalysts is a common ground in nano-technology and bio-technology and forms one of nano-biotechnology's main fields. Immobilization of enzyme, provides the possibility of transforming nano structures proposed as nano-bioreactors into molecular machines. Designed nano-bioreactor has the capability of continuous reactions. In this system, enzymes are immobilized on nano-spaces, optimum performance conditions are assessed and used in final step.

With applying continuous triple enzyme reaction, urea converted into a biologically valuable product. In first stage, each enzyme is immobilized on a cellulose nano-fiber at an optimum condition and then used in continuous reactions. In first reaction, with using urease, urea changes into NH₄⁺,

(NH₂)₂CO+3H₂O->HCO₃⁻+2NH₄⁺+OH⁻

α-Ketoglutarate+NH₄⁺+ATP+NADPH->L-Glutamate+NADP+ADP+Pi

Then with bio-mimetic from nitrogen uptake metabolic pathway and adding α-Ketoglutarate into the media, with using glutamate dehydrogenize enzyme, glutamate can be produced. Glutamate is a bio-valuable product nevertheless, with aid of next stage, added value and efficiency of this product can be raised.

α-Ketoglutarate+NH₄⁺+ATP+NADPH->L-Glutamate+NADP+ADP+Pi

In the last stage, in presence of glutamine synthetase, glutamate converted into glutamine,

Glutamate+NH₄⁺+ATP->Glutamine+ADP+Pi+H⁺

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions.

Although specific terms may be employed herein, they are used only in generic and descriptive sense and not for purposes of limitation. Accordingly, the present invention is not limited to the specific embodiments illustrated herein. While the above is a complete description of the preferred embodiments of the invention, various alternatives, modifications, and equivalents may be used. Therefore, the above description and the examples should not be taken as limiting the scope of the invention, which is defined by the appended claims.

1. An enzymatic nano-bioreactor, comprising:
at least one enzyme, and
a nano-fiber,
wherein the enzyme is a nanoparticle configured to immobilize on the nano-fiber.

2. The enzymatic nano-bioreactor of claim 1, wherein the enzyme is any one of a urease, a glutamate dehydrogenase, and a glutamine synthase.

3. The enzymatic nano-bioreactor of claim 1, wherein the nano-fiber is a bacterial cellulose nano-fiber.

4. The enzymatic nano-bioreactor of claim 1, wherein the enzyme is immobilized by crosslinking mechanism to the nanofibers.

5. The enzymatic nano-bioreactors of claim 1, wherein the enzyme comprising urease nanoparticle configured to immobilize on the nano-fiber to form a urease nano-bioreactor.

6. The enzymatic nano-bioreactors of claim 1, wherein the enzyme comprising glutamate dehydrogenase nanoparticle is configured to immobilize on the nano-fiber to form a glutamate dehydrogenase nano-bioreactor.

7. The enzymatic nano-bioreactors of claim 1, wherein the enzyme comprising glutamine synthetase nanoparticle configured to immobilize on the nano-fiber to form a glutamine synthetase nano-bioreactor.

8. The enzymatic nano-bioreactor of claim 1, wherein the optimized pH and temperature for immobilizing urease on the nano-fiber is 6.5 and about 50° C., and the optimized pH and temperature for immobilizing glutamate dehydrogenase on the nano-fiber is 8.5 and about 50° C., and the optimized pH and temperature for immobilizing glutamine synthetase on the nano-fiber is 7.5 and about 60° C.

9. An enzymatic nano-bioreactor apparatus, comprising:
a plurality of input pumps configured to introduce at least one input to a plurality of micro-bioreactors connected each other by a connector, and
at least one nano-bioreactor placed inside the micro-bioreactor, configured to receive the input, wherein the nano-bioreactor comprising:
at least one enzyme, and
a nano-fiber,
wherein the enzyme is a nanoparticle configured to immobilize on the nano-fiber, and
an outlet configured to extrude a resultant output from the nano-bioreactor to a collector unit.

10. The enzymatic nano-bioreactor apparatus of claim 9, wherein the input is urea and the resultant output is glutamine.

11. The enzymatic nano-bioreactor apparatus of claim 9, wherein the plurality of micro-bioreactors are connected to each other by a silicon connector.

12. The enzymatic nano-bioreactor apparatus of claim 9, wherein the enzyme is any one of a urease, a glutamate dehydrogenase, and a glutamine synthetase.

13. The enzymatic nano-bioreactor apparatus of claim 9, wherein the nano-fiber is a cellulose nano-fiber.

14. The enzymatic nano-bioreactor apparatus of claim 9, wherein the enzyme comprising urease nanoparticle configured to immobilize on the nano-fiber to form a urease nano-bioreactor.

15. The enzymatic nano-bioreactor apparatus of claim 9, wherein the enzyme comprising glutamate dehydrogenase nanoparticle configured to immobilize on the nano-fiber to form a glutamate dehydrogenase nano-bioreactor.

16. The enzymatic nano-bioreactor apparatus of claim 9, wherein the enzyme is immobilized by crosslinking mechanism to the nanofibers.

17. The enzymatic nano-bioreactor apparatus of claim 9, wherein the input urea is converted to ammonium by the urease nano-bioreactor, and the ammonium is converted to L-Glutamate by the glutamate dehydrogenase nano-bioreactor, and the L-Glutamate is converted to output glutamine by the glutamine synthetase nano-bioreactor.

18. A method for manipulating extracellular metabolic system, comprising:
introducing an input via a plurality of input pumps to one or more micro-bioreactors connected each other by a connector;
receiving the input by a first nano-bioreactor placed inside the first micro-bioreactor to produce first output;
receiving the first output by a second nano-bioreactor placed inside the second micro-bioreactor to produce second output;
receiving the second output by a third nano-bioreactor placed inside the third micro-bioreactor to produce final output, and
extruding and collecting the final output via an outlet to a collector unit,
wherein the plurality of micro-bioreactors are connected each other by a silicon connector,
wherein the second nano-bioreactor comprises glutamate dehydrogenase enzyme nanoparticles immobilized on the nano-fiber to form a glutamate dehydrogenase nano-bioreactor, and the third nano-bioreactor comprises glutamine synthetase enzyme nanoparticles immobilized on the nano-fiber to form a glutamine synthetase nano-bioreactor.

19. The method of claim 18, wherein the enzyme is immobilized by crosslinking mechanism to the nanofibers.

20. The method of claim 18, wherein the first output is ammonium, the second output is L-Glutamate, and the final output is glutamine.
